(19) United States
(12) Patent Application Publication
(10) Pub. No.: US 2013/0158589 A1
(43) Pub. Date: Jun. 20, 2013

(54) Methods And Kits For Treating Lacerations And Puncture Wounds Using Inverse Thermosensitive Polymers

(71) Applicant: Pluromed, Inc., Woburn, MA (US)

(72) Inventors: William E. Cohn, Bellaire, TX (US); Jean-Marie Vogel, Lincoln, MA (US)

(73) Assignee: PLUROMED, INC., Woburn, MA (US)

(21) Appl. No.: 13/766,715

(22) Filed: Feb. 13, 2013

Related U.S. Application Data
(63) Continuation of application No. 13/272,025, filed on Oct. 12, 2011, now abandoned.
Application No. 13/272,025 is a division of application No. 11/640,827, filed on Dec. 18, 2006.
(60) Provisional application No. 60/753,319, filed on Dec. 22, 2005.

(51) Int. Cl. A61K 31/765; A61K 31/785; A61M 29/02; A61K 45/06

(52) U.S. Cl. CPC A61K 31/765; A61K 45/06; A61K 31/785; A61M 29/02
USPC 606/191; 424/78.06

(57) ABSTRACT

In certain embodiments, the present invention relates to methods and kits for treating wounds, comprising the step of introducing into said wound a composition comprising at least one optionally purified inverse thermosensitive polymer, wherein said at least one optionally purified inverse thermosensitive polymer forms a gel in said wound, thereby temporarily occluding said wound. In certain embodiments, the present invention relates to the aforementioned method wherein a wound to a blood vessel or a segment of the GI tract is occluded, thereby preventing exsanguination and/or septicemia. In other embodiments, the inventive methods and kits described herein may be used to ameliorate (e.g., fill) temporarily a defect in a biological lumen, thereby strengthening said defect, preventing rupture of, or maintaining, improving or optimizing fluid flow through said lumen.

### RELATED APPLICATIONS

This application is a continuation of U.S. application Ser. No. 13/272,025, filed Oct. 12, 2011, which is a divisional of U.S. application Ser. No. 11/640,827, filed Dec. 18, 2006, which claims the benefit of U.S. Provisional Application No. 60/753,319 filed on Dec. 22, 2005. The entire teachings of the above applications are incorporated herein by reference.

### BACKGROUND OF THE INVENTION

The most common battlefield injuries involve hemorrhagic shock from bullet wounds or explosive devices with shrapnel. Hemorrhage associated with these wounds is the largest preventable cause of death among U.S. soldiers in combat, historically accounting for roughly half of all such fatalities. Until recently, techniques for controlling bleeding hadn't improved substantially since the Civil War. Typically, a medic or a fellow soldier would slap on a cotton gauze bandage while elevating and compressing a wound—a chancy procedure in the best of circumstances, and particularly trying in the face of enemy fire. Casualty numbers in current conflicts underscore the need to reduce this death rate by applying innovative products and methods. Unfortunately, most recent development focuses on improving traditional coated-bandages that are used to promote clotting (e.g., coated with fibrin, thrombin or chitosan).

According to the U.S. Army Medical Research and Material Command (USAMRMC), military casualties may wait for hours before definitive health care can be provided, initial treatment and subsequent evacuation occur in austere environments characterized by limited supplies and limited diagnostic and life-support equipment, and provision of acute and critical care is labor intensive and must frequently be provided by non-physician medical personnel. Current modalities for treating mass hemorrhaging in the field include the sprinkling on the wound of clotting agents to speed up the formation of a clot. These modalities are limited in many ways, most notably by the fact that they do not resist pressure. As a result, they are ineffective for continuously spurting blood from even a small puncture.

Recently introduced in combat is the QuickClot® blood-clotting agent from Z-Medica, which was approved by the FDA in September 2004. Although QuickClot® operates differently than a bandage (i.e., it is sprinkled on a wound to speed up the formation of a clot by removing water from the blood and thereby concentrating the clotting factors), it suffers from several drawbacks: its action is not fully reversible; it can generate substantial heat upon application; and, most importantly, it does not resist pressure. Therefore, QuickClot® is generally ineffective for continuously spurting wounds.

Accordingly, there remains an urgent need for an inexpensive, fully reversible, non-thrombogenic method to treat injuries (e.g., through the occlusion of large arteries) and associated kits, thereby allowing the safe transport of a injured person to a medical facility. Remarkably, disclosed herein are such kits and methods using inverse thermosensitive polymers.

### SUMMARY OF THE INVENTION

In certain embodiments the present invention relates to methods and kits for treating wounds (e.g., lacerations and puncture wounds), comprising the step of introducing into a wound a composition comprising at least one optionally purified inverse thermosensitive polymer, wherein said at least one optionally purified inverse thermosensitive polymer forms a gel in said wound, thereby temporarily occluding said wound preventing exsanguination and/or septicemia. In other embodiments, the inventive methods and kits described herein may be used to ameliorate (e.g., fill) temporarily a defect in a biological lumen, thereby strengthening said defect, preventing rupture of, or maintaining, improving or optimizing fluid flow through said lumen. The kits and methods of the present invention may also be used in connection with temporarily filling partially or completely an internal cavity of a mammal. The kits and methods of the present invention may also be used in connection with temporarily ameliorating a defect in a surface of a lumen.

### BRIEF DESCRIPTION OF FIGURES

FIG. 1 depicts a graph of viscosity as a function of temperature for various solutions of purified poloxamer 407.

### DETAILED DESCRIPTION OF THE INVENTION

In certain embodiments, the inventive methods and kits described herein serve to improve the care and survivability of injuries that, if left untreated, would result in severe blood loss. The inverse thermosensitive polymers of the invention may be used as a sterile, traumatic wound treatment that will rapidly arrest high-volume blood loss and achieve hemostasis in large wounds, arresting the hemorrhage before the casualty goes into shock. Importantly, the inverse thermosensitive polymers of the invention are safe to leave in the wound until the injury can be treated in a hospital or other medical facility. In certain embodiments, the method is effective in moderate-to-severe wounds, including high-volume venous and arterial bleeding. In other embodiments, the inventive methods and kits described herein may be used to ameliorate (e.g., fill) temporarily a defect in a biological lumen, thereby strengthening said defect, preventing rupture of, or maintaining, improving or optimizing fluid flow through said lumen.

### Selected Applications

The inverse thermosensitive polymer compositions of the invention may be used for all types of wound treatment and wound healing. In particular, they are useful for sealing of internal and external wounds, for securing sutures, and for healing of large-surfaced wounds or wound cavities. They are also particularly suitable for use in large or small bone cavities, of surgical or traumatic origin, in which the stoppage of bleeding is often a great problem, for example after or due to dental extractions, otological surgery fractures, punctures, lacerations or gunshot wounds.

In one application, the inventive methods have immense potential for treating soldiers suffering from hemorrhagic shock. The inverse thermosensitive polymer may be carried in a container that may be a syringe with a disposable 27-gauge safety-tipped needle, or a cannula, or a flexible plastic bag with an opening, with a special plastic exterior sheath containing ammonium nitrate and water separated by a frangible seal. Promptly post injury, the wounded individual (e.g., soldier), or someone assisting him or her, can squeeze the device, breaking the frangible seal and causing the ammonium nitrate to mix with the water. Within seconds, the device (or part of it) will become ice cold at which temperature the polymer will be a liquid. This classes of embodiments are particularly important in warm climates. The safety tip is then removed from the needle, cannula or other opening, and the needle, cannula or other kind of outlet of the device is inserted directly into the gaping wound. The polymer is discharged into the wound where, within seconds of entry, the liquid polymer becomes a firm gel, creating a safe and reversible occlusion. In certain embodiments, the occlusion will last about 120 minutes, about 60 minutes, or 10 to 45 minutes, depending on the amount applied; the material may be reapplied as needed to provide occlusion for longer periods.

In another application, the inventive methods and kits may be used for treatment of civilian gunshot wounds while the injured person is being transported to a hospital or other medical facility.

In another related application, the inventive methods may be used for the prevention, management, reduction and control of internal infection resulting from pierced or lacerated lumen in a mammal; for example, a pierced or lacerated gastrointestinal (GI) tract in a human. When deployed, the inverse thermosensitive polymer will help maintain or restore the integrity of the GI tract, and help maintain the contents in place, thereby ceasing, minimizing, or preventing spillage into the peritoneal cavity of the body. Additionally, in this and other embodiments, the product may contain a therapeutic agent, such as one or more broad-spectrum antibiotics. Importantly, the method of preparation of the product allows incorporation of therapeutic agents at a wide range of concentrations.

Intra-abdominal infections are among the most difficult infections to treat effectively. A successful outcome depends on early diagnosis, rapid and appropriate intervention, and selection of efficacious antibiotic regimens. Mortality rates associated with intra-abdominal infections range from 3.5% in patients with early infection following penetrating abdominal trauma, to more than 60% in patients with well-established infection coupled with resultant multiple organ failure. These deep-seated infections generally occur after the continuity of the GI tract is interrupted by trauma (e.g., an abdominal gunshot wound). The leakage of the endogenous microflora of the GI tract into adjacent tissues appears to overwhelm the patient's defense mechanisms, resulting in infection. When dissemination of the microflora is controlled and the initial event is promptly treated with appropriate intervention and parenteral antibiotics, the chance for subsequent localized abscess decreases significantly. Consequently, use of the methods and kits including one or more antibiotics has the potential to prevent or minimize peritoneal infection in a patient with a lacerated or punctured GI tract.

In other embodiments, the inventive methods may be used to ameliorate (e.g., fill) temporarily a defect in a biological lumen, thereby strengthening said defect, preventing rupture of, or maintaining, improving or optimizing fluid flow through said lumen. For example, an aneurism in a lumen could be filled to strengthen it and potentially prevent it from bursting.

### Kits

This invention also provides kits for conveniently and effectively implementing the methods of this invention. Such kits comprise any of the block copolymers of the present invention or a combination thereof, and a means for facilitating their use consistent with methods of this invention. Such kits may also included ice, a cold pack, or other means of cooling. Such kits provide a convenient and effective means for assuring that the methods are practiced in an effective manner. The compliance means of such kits includes any means which facilitates practicing a method of this invention. Such compliance means include instructions, packaging, and dispensing means, and combinations thereof. Kit components may be packaged for either manual or partially or wholly automated practice of the foregoing methods. In other embodiments, this invention contemplates a kit including block copolymers of the present invention, and optionally instructions for their use. In certain embodiments, the inverse thermosensitive copolymers of such a kit of the present invention are contained in one or more syringes, a compressible plastic or metal tube (for example, akin to a conventional toothpaste tube), a packet that may be torn open, or a blister pack that may be broken in proximity to a wound.

### Selected Therapeutic Agents

The reversibly gelling polymers used in the methods of the invention have physico-chemical characteristics that make them suitable delivery vehicles for conventional small-molecule drugs, as well as macromolecular (e.g., peptides) drugs or other therapeutic products. Therefore, the composition comprising the thermosensitive polymer may further comprise a pharmaceutic agent selected to provide a pre-selected pharmaceutic effect. A pharmaceutic effect is one which seeks to prevent or treat the source or symptom of a disease or physical disorder. Pharmaceutics include those products subject to regulation under the FDA pharmaceutic guidelines. Importantly, the compositions used in methods of the invention are capable of solubilizing and releasing bioactive materials. Solubilization is expected to occur as a result of dissolution in the bulk aqueous phase or by incorporation of the solute in micelles created by the hydrophobic domains of the poloxamer. Release of the drug would occur through diffusion or network erosion mechanisms.

Those skilled in the art will appreciate that the compositions used in the methods of the invention may simultaneously be utilized to deliver a wide variety of pharmaceutics to a wound site. To prepare a pharmaceutic composition, an effective amount of pharmaceutically active agent(s), which imparts the desirable pharmaceutic effect is incorporated into the reversibly gelling composition used in the methods of the invention. Preferably, the selected agent is water soluble, which will readily lend itself to a homogeneous dispersion throughout the reversibly gelling composition. It is also preferred that the agent(s) is non-reactive with the composition. For materials, which are not water soluble, it is also within the scope of the methods of the invention to disperse or suspend lipophilic material throughout the composition. Myriad bioactive materials may be delivered using the methods of the present invention; the delivered bioactive material includes anesthetics, antimicrobial agents (antibacterial, antifungal, antiviral), anti-inflammatory agents, diagnostic agents, and wound-healing agents.

Because the reversibly gelling composition used in the methods of the present invention are suited for application under a variety of environmental conditions, a wide variety of pharmaceutically active agents may be incorporated into and administered via the composition. The pharmaceutic agent loaded into the polymer networks of the thermosensitive polymer may be any substance having biological activity, including proteins, polypeptides, polynucleotides, nucleoproteins, polysaccharides, glycoproteins, lipoproteins, and synthetic and biologically engineered analogs thereof.

A vast number of therapeutic agents may be incorporated in the polymers used in the methods of the present invention. In general, therapeutic agents which may be administered via the methods of the invention include, without limitation: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins. Suitable pharmaceuticals for parenteral administration are well known as is exemplified by the Handbook on Injectable Drugs, 6th Edition, by Lawrence A. Trissel, American Society of Hospital Pharmacists. Bethesda, Md., 1990 (hereby incorporated by reference).

The pharmaceutically active compound may be any substance having biological activity, including proteins, polypeptides, polynucleotides, nucleoproteins, polysaccharides, glycoproteins, lipoproteins, and synthetic and biologically engineered analogs thereof. The term “protein” is art-recognized and for purposes of this invention also encompasses peptides. The proteins or peptides may be any biologically active protein or peptide, naturally occurring or synthetic.

Examples of proteins include antibodies, enzymes, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The pharmaceutical agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelop glycoproteins and cytokins and other agents selected from the group consisting of bovine somatropine (sometimes referred to as BST), estrogens, androgens, insulin growth factors (sometimes referred to as IGF), interleukin I, interleukin II and cytokins. Three such cytokins are interferon-β, interferon-γ and tuftsin.
Examples of bacterial toxoids that may be incorporated in the compositions used in the methods of the invention are tetanus, diphtheria, pseudomonas A, mycobaeterium tuberculosis. Examples of that may be incorporated in the compositions used in the occlusion methods of the invention are HIV envelope glycoproteins, e.g., gp120 or gp 160, for AIDS vaccines. Examples of anti-ulcer H2 receptor antagonists that may be included are ranitidine, cimetidine and famotidine, and other anti-ulcer drugs are omparazide, cesupride and misoprostol. An example of a hypoglycaemic agent is glizipide.

Classes of pharmaceutically active compounds which can be loaded into that may be incorporated in the compositions used in the occlusion methods of the invention include, but are not limited to, anti-AIDS substances, anti-cancer substances, antibiotics, immunosuppressants (e.g., cyclosporine) anti-viral substances, enzyme inhibitors, neurotoxins, opioids, hypnotics, antihistamines, lubricants tranquilizers, anti-convulsants, muscle relaxants and anti-Parkinson substances, anti-spasmodics and muscle contractants, miotics and anti-cholinergics, anti-glaucoma compounds, anti-parasite and/or anti-protozoal compounds, anti-hypertensives, analgesics, anti-pyretics and anti-inflammatory agents such as NSAIDs, local anesthetics, ophthalmics, prostaglandins, anti-depressants, anti-psychotic substances, anti-emetics, imaging agents, specific targeting agents, neurotransmitters, proteins, cell response modifiers, and vaccines.

Exemplary pharmaceutical agents considered to be particularly suitable for incorporation in the compositions used in the methods of the invention include but are not limited to imidazoles, such as miconazole, econazole, terconazole, saperconazole, itraconazole, metronidazole, fluconazole, ketoconazole, and clotrimazole, luteinizing-hormone-releasing hormone (LHRH) and its analogues, nonoxynol-9, a GnRH agonist or antagonist, natural or synthetic progestrin, such as selected progesterone, 17-hydroxyprogeterone derivatives such as medroxyprogesterone acetate, and 19-nortestosterone analogues such as norethindrone, natural or synthetic estrogens, conjugated estrogens, estradiol, estropipate, and ethinyl estradiol, bisphosphonates including etidronate, alendronate, tiludronate, resedronate, clodronate, and pamidronate, calcitonin, parathyroid hormones, carbonic anhydrase inhibitor such as felbamate and dorzolamide, a mast cell stabilizer such as xesterbergsterol-A, lodoxamine, and cromolyn, a prostaglandin inhibitor such as diclofenac and ketorolac, a steroid such as prednisolone, dexamethasone, fluoromethylone, rimexolone, and lotepednol, an antihistamine such as antazoline, pheniramine, and histiminase, pilocarpine nitrate, a beta-blocker such as levobunolol and timolol maleate. As will be understood by those skilled in the art, two or more pharmaceutical agents may be combined for specific effects. The necessary amounts of active ingredient can be determined by simple experimentation.

By way of example only, any of a number of antibiotics and antimicrobials may be included in the thermosensitive polymers used in the methods of the invention. Antimicrobial drugs preferred for inclusion in compositions used in the occlusion methods of the invention include salts of lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole and amanfadine and the like.

By way of example only, in the case of anti-inflammation, non-steroidal anti-inflammatory agents (NSAIDS) may be incorporated in the compositions used in the occlusion methods of the invention, such as propionic acid derivatives, acetic acid, fenamic acid derivatives, biphenylcarboxylic acid derivatives, oxicams, including but not limited to aspirin, acetaminophen, ibuprofen, naproxen, benoxaprofen, flurbiprofen, fenbufen, ketoprofen, indoprofen, pirprofen, carporfen, and bucloxic acid and the like.

### Selected Methods of the Present Invention

In certain embodiments, the present invention relates to a method of occluding a wound in a mammal, comprising the step of introducing into said wound a composition comprising at least one optionally purified inverse thermosensitive polymer, wherein said at least one optionally purified inverse thermosensitive polymer forms a gel in said wound; thereby temporarily occluding said wound.

In certain embodiments, the present invention relates to a method of partially or completely filling an internal cavity in a mammal, comprising the step of introducing into said internal cavity a composition comprising at least one optionally purified inverse thermosensitive polymer, wherein said at least one optionally purified inverse thermosensitive polymer forms a gel in said cavity, thereby temporarily filling partially or completely said internal cavity.

In certain embodiments, the present invention relates to a method of ameliorating a defect in a surface of a lumen in a mammal, comprising the step of introducing into said defect in the surface of a lumen a composition comprising at least one optionally purified inverse thermosensitive polymer, wherein said at least one optionally purified inverse thermosensitive polymer forms a gel in said defect in the surface of a lumen, thereby temporarily ameliorating said defect in the surface of a lumen.

In certain embodiments, the present invention relates to any one of the aforementioned methods and any of the attendant limitations, wherein said at least one optionally purified inverse thermosensitive polymer is selected from the group consisting of block copolymers, random copolymers, graft polymers, and branched copolymers.

In certain embodiments, the present invention relates to any one of the aforementioned methods and any of the attendant limitations, wherein said at least one optionally purified inverse thermosensitive polymer is a polyoxyalkylene block copolymer.

In certain embodiments, the present invention relates to any one of the aforementioned methods and any of the attendant limitations, wherein said at least one optionally purified inverse thermosensitive polymer is selected from the group consisting of poloxamers and poloxamines.

In certain embodiments, the present invention relates to any one of the aforementioned methods and any of the attendant limitations, wherein said at least one optionally purified inverse thermosensitive polymer is selected from the group consisting of poloxamer 407, poloxamer 338, poloxamer 118, Tetronic® 1107 or Tetronic® 1307.

In certain embodiments, the present invention relates to any one of the aforementioned methods and any of the attendant limitations, wherein said at least one optionally purified inverse thermosensitive polymer is poloxamer 407.

In certain embodiments, the present invention relates to any one of the aforementioned methods and any of the attendant limitations, wherein said at least one optionally purified inverse thermosensitive polymer is selected from the group consisting of purified poloxamers and purified poloxamines.

In certain embodiments, the present invention relates to any one of the aforementioned methods and any of the attendant limitations, wherein said at least one optionally purified inverse thermosensitive polymer is selected from the group consisting of purified poloxamer 407, purified poloxamer 338, purified poloxamer 118, purified Tetronic® 1107 or purified Tetronic® 1307.

In certain embodiments, the present invention relates to any one of the aforementioned methods and any of the attendant limitations, wherein said at least one optionally purified inverse thermosensitive polymer is purified poloxamer 407.

In certain embodiments, the present invention relates to any one of the aforementioned methods and any of the attendant limitations, wherein said composition has a transition temperature of between about 10° C. and about 40° C.

In certain embodiments, the present invention relates to any one of the aforementioned methods and any of the attendant limitations, wherein said composition has a transition temperature of between about 15° C. and about 30° C.

In certain embodiments, the present invention relates to any one of the aforementioned methods and any of the attendant limitations, wherein the volume of said composition at physiological temperature is about 80% to about 120% of its volume below its transition temperature.

In certain embodiments, the present invention relates to any one of the aforementioned methods and any of the attendant limitations, wherein the volume of said composition at physiological temperature is about 80% to about 120% of its volume below its transition temperature; and said composition has a transition temperature of between about 10° C. and about 40° C.

In certain embodiments, the present invention relates to any one of the aforementioned methods and any of the attendant limitations, wherein the volume of said composition at physiological temperature is about 80% to about 120% of its volume below its transition temperature; and said composition has a transition temperature of between about 15° C. and about 30° C.

In certain embodiments, the present invention relates to any one of the aforementioned methods and any of the attendant limitations, wherein the volume of said composition at physiological temperature is about 80% to about 120% of its volume below its transition temperature; said composition has a transition temperature of between about 10° C. and about 40° C.; and said composition comprises at least one optionally purified inverse thermosensitive polymer selected from the group consisting of poloxamers and poloxamines.

In certain embodiments, the present invention relates to any one of the aforementioned methods and any of the attendant limitations, wherein the volume of said composition at physiological temperature is about 80% to about 120% of its volume below its transition temperature; said composition has a transition temperature of between about 15° C. and about 30° C.; and said composition comprises at least one optionally purified inverse thermosensitive polymer selected from the group consisting of poloxamers and poloxamines.

In certain embodiments, the present invention relates to any one of the aforementioned methods and any of the attendant limitations, wherein said composition comprises about 5% to about 35% of said inverse thermosensitive polymer.

In certain embodiments, the present invention relates to any one of the aforementioned methods and any of the attendant limitations, wherein said composition comprises about 10% to about 30% of said inverse thermosensitive polymer.

In certain embodiments, the present invention relates to any one of the aforementioned methods and any of the attendant limitations, wherein said at least one optionally purified inverse thermosensitive polymer has a polydispersity index from about 1.5 to about 1.0.

In certain embodiments, the present invention relates to any one of the aforementioned methods and any of the attendant limitations, wherein said at least one optionally purified inverse thermosensitive polymer has a polydispersity index from about 1.2 to about 1.0.

In certain embodiments, the present invention relates to any one of the aforementioned methods and any of the attendant limitations, wherein said composition occludes said wound, fills said cavity or ameliorates said defect for about thirty minutes.

In certain embodiments, the present invention relates to any one of the aforementioned methods and any of the attendant limitations, wherein said composition occludes said wound, fills said cavity or ameliorates said defect for about forty-five minutes.

In certain embodiments, the present invention relates to any one of the aforementioned methods and any of the attendant limitations, wherein said composition occludes said wound, fills said cavity or ameliorates said defect for about one hour.

In certain embodiments, the present invention relates to any one of the aforementioned methods and any of the attendant limitations, wherein said composition further comprises a therapeutic agent.

In certain embodiments, the present invention relates to any one of the aforementioned methods and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to any one of the aforementioned methods and any of the attendant limitations, wherein the therapeutic agent is an antibiotic.

In certain embodiments, the present invention relates to any one of the aforementioned methods and any of the attendant limitations, wherein said mammal is a human.

In certain embodiments, the present invention relates to any one of the aforementioned methods and any of the attendant limitations, wherein said composition is introduced to said wound, cavity or defect using a syringe, cannula, tube, packet, or catheter.

In certain embodiments, the present invention relates to any one of the aforementioned methods and any of the attendant limitations, wherein said composition is introduced to said wound, cavity or defect using a syringe or tube.

In certain embodiments, the present invention relates to any one of the aforementioned methods and any of the attendant limitations, further comprising the step of cooling said composition prior to introduction into said wound, cavity or defect.

In certain embodiments, the present invention relates to any one of the aforementioned methods and any of the attendant limitations, wherein said composition is cooled to about 15° C.

In certain embodiments, the present invention relates to any one of the aforementioned methods and any of the attendant limitations, wherein said composition is cooled to about 10° C.

In certain embodiments, the present invention relates to any one of the aforementioned methods and any of the attendant limitations, wherein said composition is cooled to about 5° C.

In certain embodiments, the present invention relates to any one of the aforementioned methods and any of the attendant limitations, wherein said composition is cooled to about 0° C.

In certain embodiments, the present invention relates to any one of the aforementioned methods and any of the attendant limitations, wherein said composition is cooled with ice, water, and/or a cold pack.

In certain embodiments, the present invention relates to any one of the aforementioned methods and any of the attendant limitations, wherein a wound is occluded; and said wound is an arterial, venous or gastrointestinal wound.

In certain embodiments, the present invention relates to any one of the aforementioned methods and any of the attendant limitations, wherein a wound is occluded; and said wound is a puncture wound.

In certain embodiments, the present invention relates to any one of the aforementioned methods and any of the attendant limitations, wherein a wound is occluded; and said wound is a gunshot wound.

In certain embodiments, the present invention relates to the aforementioned methods and any of the attendant limitations, further comprising the step of placing an elastomeric balloon into said wound, internal cavity or defect; wherein said composition comprising at least one optionally purified inverse thermosensitive polymer is introduced into said balloon, thereby inflating said balloon. One advantage of such a method (i.e., one which employs an elastometic balloon) is that the thermosensitive polymer composition does not come into direct contact with the subject. In addition, the use of an elastomeric balloon may aid in both administration and removal of said thermosensitive polymer composition.

The aforementioned elastometic balloon may be made of any suitable, biocompatible material. In certain embodiments, the present invention relates to the aforementioned method and any of the attendant limitations, wherein said elastomeric balloon is made from polyethylenes, polyamides, polyurethanes, latexes or silicone rubbers.

In certain embodiments, the present invention relates to the aforementioned methods and any of the attendant limitations, wherein the elastomeric balloon has a balloon-wall thickness in the range from 0.025 mm to 0.25 mm. In certain embodiments, the present invention relates to the aforementioned methods and any of the attendant limitations, wherein the elastomeric balloon has a balloon-wall thickness greater than about 0.25 mm.

In certain embodiments, the present invention relates to the aforementioned methods and any of the attendant limitations, wherein the elastomeric balloon is inflated to an internal pressure in the range from 1 psi to 60 psi.

In certain embodiments, the present invention relates to the aforementioned methods and any of the attendant limitations, wherein the elastomeric balloon is inflated with a volume of said thermosensitive polymer composition of from about 1 mL to 500 mL. In certain embodiments, the present invention relates to the aforementioned methods and any of the attendant limitations, wherein the elastomeric balloon is inflated with a volume of said thermosensitive polymer composition of from about 10 mL to 100 mL. In certain embodiments, the present invention relates to the aforementioned methods and any of the attendant limitations, wherein the elastomeric balloon is inflated with a volume of said thermosensitive polymer composition greater than 1 mL.

In certain embodiments, the balloon may be placed in the subject via a balloon catheter. For example, a balloon catheter may be used, said catheter comprising a catheter body having a proximal end, a distal end, and at least one inflation lumen therethrough; and an elastomeric balloon disposed over the distal end of the catheter body to receive inflation medium from the inflation lumen.

### Selected Kits of the Present Invention

In certain embodiments, the present invention relates to a kit, comprising instructions for use thereof; and a composition comprising at least one optionally purified inverse thermosensitive polymer, wherein said inverse thermosensitive polymer is a gel at mammalian physiological temperature.

In certain embodiments, the present invention relates to the aforementioned kit and any of the attendant limitations, wherein said composition is contained in a packet or tube.

In certain embodiments, the present invention relates to the aforementioned kit and any of the attendant limitations, further comprising a cold pack.

In certain embodiments, the present invention relates to the aforementioned kit and any of the attendant limitations, further comprising a syringe or cannula.

In certain embodiments, the present invention relates to the aforementioned kit and any of the attendant limitations, wherein said composition further comprises a therapeutic agent.

In certain embodiments, the present invention relates to the aforementioned kit and any of the attendant limitations, wherein the therapeutic agent is selected from the group consisting of antiinflammatories, antibiotics, antimicrobials, chemotherapeutics, antivirals, analgesics, and antiproliferatives.

In certain embodiments, the present invention relates to the aforementioned kit and any of the attendant limitations, wherein the therapeutic agent is an antibiotic.

In certain embodiments, the present invention relates to the aforementioned kit and any of the attendant limitations, wherein said at least one optionally purified inverse thermosensitive polymer is selected from the group consisting of block copolymers, random copolymers, graft polymers, and branched copolymers.

In certain embodiments, the present invention relates to the aforementioned kit and any of the attendant limitations, wherein said at least one optionally purified inverse thermosensitive polymer is a polyoxyalkylene block copolymer.

In certain embodiments, the present invention relates to the aforementioned kit and any of the attendant limitations, wherein said at least one optionally purified inverse thermosensitive polymer is selected from the group consisting of poloxamers and poloxamines.

In certain embodiments, the present invention relates to the aforementioned kit and any of the attendant limitations, wherein said at least one optionally purified inverse thermosensitive polymer is selected from the group consisting of poloxamer 407, poloxamer 338, poloxamer 118, Tetronic® 1107 or Tetronic® 1307.

In certain embodiments, the present invention relates to the aforementioned kit and any of the attendant limitations, wherein said at least one optionally purified inverse thermosensitive polymer is poloxamer 407.

In certain embodiments, the present invention relates to the aforementioned kit and any of the attendant limitations, wherein said at least one optionally purified inverse thermosensitive polymer is selected from the group consisting of purified poloxamers and purified poloxamines.

In certain embodiments, the present invention relates to the aforementioned kit and any of the attendant limitations, wherein said at least one optionally purified inverse thermosensitive polymer is selected from the group consisting of purified poloxamer 407, purified poloxamer 338, purified poloxamer 118, purified Tetronic® 1107 or purified Tetronic® 1307.

In certain embodiments, the present invention relates to the aforementioned kit and any of the attendant limitations, wherein said at least one optionally purified inverse thermosensitive polymer is purified poloxamer 407.

In certain embodiments, the present invention relates to the aforementioned kit and any of the attendant limitations, wherein said composition has a transition temperature of between about 10° C. and about 40° C.

In certain embodiments, the present invention relates to the aforementioned kit and any of the attendant limitations, wherein said composition has a transition temperature of between about 15° C. and about 30° C.

In certain embodiments, the present invention relates to the aforementioned kit and any of the attendant limitations, wherein the volume of said composition at physiological temperature is about 80% to about 120% of its volume below its transition temperature.

In certain embodiments, the present invention relates to the aforementioned kit and any of the attendant limitations, wherein the volume of said composition at physiological temperature is about 80% to about 120% of its volume below its transition temperature; and said composition has a transition temperature of between about 10° C. and about 40° C.

In certain embodiments, the present invention relates to the aforementioned kit and any of the attendant limitations, wherein the volume of said composition at physiological temperature is about 80% to about 120% of its volume below its transition temperature; and said composition has a transition temperature of between about 15° C. and about 30° C.

In certain embodiments, the present invention relates to the aforementioned kit and any of the attendant limitations, wherein the volume of said composition at physiological temperature is about 80% to about 120% of its volume below its transition temperature; said composition has a transition temperature of between about 10° C. and about 40° C.; and said composition comprises at least one optionally purified inverse thermosensitive polymer selected from the group consisting of poloxamers and poloxamines.

In certain embodiments, the present invention relates to the aforementioned kit and any of the attendant limitations, wherein the volume of said composition at physiological temperature is about 80% to about 120% of its volume below its transition temperature; said composition has a transition temperature of between about 15° C. and about 30° C.; and said composition comprises at least one optionally purified inverse thermosensitive polymer selected from the group consisting of poloxamers and poloxamines.

In certain embodiments, the present invention relates to the aforementioned kit and any of the attendant limitations, wherein said composition comprises about 5% to about 35% of said inverse thermosensitive polymer.

In certain embodiments, the present invention relates to the aforementioned kit and any of the attendant limitations, wherein said composition comprises about 10% to about 30% of said inverse thermosensitive polymer.

In certain embodiments, the present invention relates to the aforementioned kit and any of the attendant limitations, wherein said at least one optionally purified inverse thermosensitive polymer has a polydispersity index from about 1.5 to about 1.0.

In certain embodiments, the present invention relates to the aforementioned kit and any of the attendant limitations, wherein said at least one optionally purified inverse thermosensitive polymer has a polydispersity index from about 1.2 to about 1.0.

In certain embodiments, the present invention relates to the aforementioned kit and any of the attendant limitations, further comprising an elastomeric balloon.

In certain embodiments, the present invention relates to the aforementioned kit and any of the attendant limitations, wherein said elastomeric balloon is made from polyethylenes, polyamides, polyurethanes, latexes or silicone rubbers.

### Inverse Thermosensitive Polymers

In general, the inverse thermosensitive polymers used in the methods of the invention, which become a gel at or about body temperature, can be administered in a liquid form. The material, upon reaching body temperature, undergoes a transition from a liquid to a gel. The inverse thermosensitive polymers used in connection with the methods of the invention may comprise a block copolymer with inverse thermal gelation properties. The block copolymer can further comprise a polyoxyethylene-polyoxypropylene block copolymer, such as a biodegradable, biocompatible copolymer of polyethylene oxide and polypropylene oxide. Also, the inverse thermosensitive polymer can include one or more additives; for example, therapeutic agents may be added to the inverse thermosensitive polymers.

In certain embodiments, the polymer composition of the invention may be a flexible or flowable material. By “flowable” is meant the ability to assume, over time, the shape of the space containing it at body temperature. This characteristic includes, for example, liquid compositions that are suitable for: spraying into a site; injection with a manually operated syringe fitted with, for example, a 23-gauge needle; or delivery through a catheter.

Also encompassed by the term “flowable” are highly viscous, gel-like materials at room temperature that may be delivered to the desired site by pouring, squeezing from a tube, or being injected with any one of the commercially available power injection devices that provide injection pressures greater than would be exerted by manual means alone. When the polymer used is itself flowable, the polymer composition of the invention, even when viscous, need not include a biocompatible solvent to be flowable, although trace or residual amounts of biocompatible solvents may be present.

In certain embodiments, the block copolymers have molecular weights ranging from about 2000 to about 1,000,000 Daltons, more particularly at least about 10,000 Daltons, and even more specifically at least about 25,000 Daltons or even at least about 50,000 Daltons. In a preferred embodiment, the block copolymers have a molecular weight between about 5,000 Daltons and about 30,000 Daltons. Number-average molecular weight (Mₙ) may also vary, but will generally fall in the range of about 1,000 to about 400,000 Daltons, preferably from about 1,000 to about 100,000 Daltons and, even more preferably, from about 1,000 to about 70,000 Daltons. Most preferably, Mₙ varies between about 5,000 and about 300,000 Daltons.

The molecular weight of the inverse thermosensitive polymer is preferably between 1,000 and 50,000, more preferably between 5,000 and 35,000. Preferably the polymer is in an aqueous solution. For example, typical aqueous solutions contain about 5% to about 30% polymer, preferably about 10% to about 25%. The molecular weight of a suitable inverse thermosensitive polymer (such as a poloxamer or poloxamine) may be, for example, between 5,000 and 25,000, and more particularly between 7,000 and 20,000.

The pH of the inverse thermosensitive polymer formulation administered to a mammal is, generally, about 6.0 to about 7.8, which are suitable pH levels for injection into the mammalian body. The pH level may be adjusted by any suitable acid or base, such as hydrochloric acid or sodium hydroxide.

In certain embodiments, the inverse thermosensitive polymers of the invention are poloxamers or poloxamines. Pluronic® polymers have unique surfactant abilities and extremely low toxicity and immunogenic responses. These products have low acute oral and dermal toxicity and low potential for causing irritation or sensitization, and the general chronic and sub-chronic toxicity is low. In fact, Pluronic® polymers are among a small number of surfactants that have been approved by the FDA for direct use in medical applications and as food additives (BASF (1990) Pluronic® Tetronic® Surfactants, BASF Co., Mount Olive, N.J.). Recently, several Pluronic® polymers have been found to enhance the therapeutic effect of drugs, and the gene transfer efficiency mediated by adenovirus. (March K L, Madison J E, Trapnell B C. “Pharmacokinetics of adenoviral vector-mediated gene delivery to vascular smooth muscle cells: modulation by poloxamer 407 and implication for cardiovascular gene therapy” Hum Gene Therapy 1995, 6, 41-53).

. Interestingly, poloxamers (or Pluronics), as nonionic surfactants, are widely used in diverse industrial applications. (Nonionic Surfactants: polyoxyalkylene block copolymers. Vol. 60. Nace V M, Dekker M (editors), New York, 1996. 280 pp.) Their surfactant properties have been useful in detergency, dispersion, stabilization, foaming, and emulsification. (Cabana A, Abdellatif A K, Juhasz J. “Study of the gelation process of polyethylene oxide. polypropylene oxide-polyethylene oxide copolymer (poloxamer 407) aqueous solutions.” Journal of Colloid and Interface Science. 1997; 190: 307-312.) Certain poloxamines, e.g., poloxamine 1307 and 1107, also display inverse thermosensitivity.

Importantly, several members of this class of polymer, poloxamer 188, poloxamer 407, poloxamer 338, poloxamines 1107 and 1307 show inverse thermosensitivity within the physiological temperature range, (Qiu Y, Park K. Environment-sensitive hydrogels for drug delivery. Adv Drug Deliv Rev. 2001, 53(3), 321-339; and Ron E S, Bromberg L E Temperature-responsive gels and thermogelling polymer matrices for protein and peptide delivery Adv Drug Deliv Rev, 1998, 31(3), 197-221.) In other words, these polymers are members of a class that are soluble in aqueous solutions at low temperature, but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. As noted above, certain poly(ethyleneoxide)/poly(propyleneoxide) block copolymers have these properties; they are available commercially as Pluronic® poloxamers and Tetronic® poloxamines (BASF, Ludwigshafen, Germany) and generically known as poloxamers and poloxamines, respectively. (See U.S. Pat. Nos. 4,188,373, 4,478,822 and 4,474,751; all of which are incorporated by reference).

The average molecular weights of commercially available poloxamers and poloxamines range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15% to about 50% of the total mass of the commercial polymer.

### Purification of Inverse Thermosensitive Polymers

The inverse thermosensitive polymers may be purified using a process for the fractionation of water-soluble polymers, comprising the steps of dissolving a known amount of the polymer in water, adding a soluble extraction salt to the polymer solution, maintaining the solution at a constant optimal temperature for a period of time adequate for two distinct phases to appear, and separating physically the phases. Additionally, the phase containing the polymer fraction of the preferred molecular weight may be diluted to the original volume with water, extraction salt may be added to achieve the original concentration, and the separation process repeated as needed until a polymer having a narrower molecular weight distribution than the starting material and optimal physical characteristics can be recovered.

In certain embodiments, a purified poloxamer or poloxamine has a polydispersity index from about 1.5 to about 1.0. In certain embodiments, a purified poloxamer or poloxamine has a polydispersity index from about 1.2 to about 1.0.

The aforementioned process consists of forming an aqueous two-phase system composed of the polymer and an appropriate salt in water. In such a system, a soluble salt can be added to a single phase polymer-water system to induce phase separation to yield a high salt, low polymer bottom phase, and a low salt, high polymer upper phase. Lower molecular weight polymers partition preferentially into the high salt, low polymer phase. Polymers that can be fractionated using this process include polyethers, glycols such as poly(ethylene glycol) and poly(ethylene oxide)s, polyoxyalkylene block copolymers such as poloxamers, poloxamines, and polyoxypropylene/polyoxybutylene copolymers, and other polyols, such as polyvinyl alcohol. The average molecular weight of these polymers may range from about 800 to greater than 100,000 Daltons. See U.S. Pat. No. 6,761,824. The aforementioned purification process inherently exploits the differences in size and polarity, and therefore solubility, among the poloxamer molecules, the poly(oxyethylene) homopolymer and the poly(oxyethylene)/poly(oxypropylene) diblock byproducts. The polar fraction of the poloxamer, which generally includes the lower molecular weight fraction and the byproducts, is removed allowing the higher molecular weight fraction of poloxamer to be recovered. The larger molecular weight poloxamer recovered by this method has physical characteristics substantially different from the starting material or commercially available poloxamer including a higher average molecular weight, lower polydispersity and a higher viscosity in aqueous solution.

Other purification methods may be used to achieve the desired outcome. For example, WO 92/16484 discloses the use of gel permeation chromatography to isolate a fraction of poloxamer 188 that exhibits beneficial biological effects, without causing potentially deleterious side effects. The copolymer thus obtained had a polydispersity index of 1.07 or less, and was substantially saturated. The potentially harmful side effects were shown to be associated with the low molecular weight, unsaturated portion of the polymer, while the medically beneficial effects resided in the uniform higher molecular weight material. Other similarly improved copolymers were obtained by purifying either the polyoxypropylene center block during synthesis of the copolymer, or the copolymer product itself (e.g., U.S. Pat. No. 5,523,492 and U.S. Pat. No. 5,696,298).

Further, a supercritical fluid extraction technique has been used to fractionate a polyoxyalkylene block copolymer as disclosed in U.S. Pat. No. 5,567,859. A purified fraction was obtained, which was composed of a fairly uniform polyoxyalkylene block copolymer having a polydispersity of less than 1.17. According to this method, the lower molecular weight fraction was removed in a stream of carbon dioxide maintained at a pressure of 2200 pounds per square inch (psi) and a temperature of 40° C.

Additionally, U.S. Pat. No. 5,800,711 discloses a process for the fractionation of polyoxyalkylene block copolymers by the batchwise removal of low molecular weight species using a salt extraction and liquid phase separation technique. Poloxamer 407 and poloxamer 188 were fractionated by this method. In each case, a copolymer fraction was obtained which had a higher average molecular weight and a lower polydispersity index as compared to the starting material. However, the changes in polydispersity index were modest and analysis by gel permeation chromatography indicated that some low-molecular-weight material remained. The viscosity of aqueous solutions of the fractionated polymers was significantly greater than the viscosity of the commercially available polymers at temperatures between 10° C. and 37° C., an important property for some medical and drug delivery applications. Nevertheless, some of the low molecular weight contaminants of these polymers are thought to cause deleterious side effects when used inside the body, making it especially important that they be removed in the fractionation process. As a consequence, polyoxyalkylene block copolymers fractionated by this process are not appropriate for all medical uses.

### DEFINITIONS

For convenience, before further description of the present invention, certain terms employed in the specification, examples, and appended claims are collected here. These definitions should be read in light of the remainder of the disclosure and understood as by a person of skill in the art.

The indefinite articles “a” and “an,” as used herein in the specification and in the claims, unless clearly indicated to the contrary, should be understood to mean “at least one,”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising.” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time.

The term “poloxamer” denotes a symmetrical block copolymer, consisting of a core of PPG polyoxyethylated to both its terminal hydroxyl groups, i.e. conforming to the interchangable generic formula (PEG)_{X}-(PPG)_{Y}-(PEG)_{X} and (PEO)_{X}-(PPO)_{Y}-(PEO)_{X}. Each poloxamer name ends with an arbitrary code number, which is related to the average numerical values of the respective monomer units denoted by X and Y.

The term “poloxamine” denotes a polyalkoxylated symmetrical block copolymer of ethylene diamine conforming to the general type [(PEG)_{X}-(PPG)_{Y}]₂—NCH₂CH₂N—[(PPG)_{Y}-(PEG)_{X}]₂. Each Poloxamine name is followed by an arbitrary code number, which is related to the average numerical values of the respective monomer units denoted by X and Y.

The term “inverse thermosensitive polymer” as used herein refers to a polymer that is soluble in water at ambient temperature, but at least partially phase-separates out of water at physiological temperature. Inverse thermosensitive polymers include poloxamer 407, poloxamer 188. Pluronic® F127. Pluronic® F68, poly(N-isopropylacrylamide), poly(methyl vinyl ether), poly(N-vinylcaprolactam); and certain poly(organophosphazenes). (Lee, B H et al. Synthesis and Characterization of Thermosensitive Poly(organophosphazenes) with Methoxy-Poly(ethylene glycol) and Alkylamines as Side Groups. Bull. Korean Chem. Soc. 2002, 23, 549-554.)

The terms “reversibly gelling” and “inverse thermosensitive” refer to the property of a polymer wherein gelation takes place upon an increase in temperature, rather than a decrease in temperature.

The term “transition temperature” refers to the temperature or temperature range at which gelation of an inverse thermosensitive polymer occurs.

The term “degradable”, as used herein, refers to having the property of breaking down or degrading under certain conditions, e.g. by dissolution.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

The term “biocompatible”, as used herein, refers to having the property of being biologically compatible by not producing a toxic, injurious, or immunological response in living tissue.

The term “lumen” denotes the space enclosed by a tube-like structure or hollow organ, such as inside an artery, a vein, a kidney, a gall bladder, a ureter, a urinary bladder, a pancreas, a salivary gland, a small intestine or a large intestine (i.e., an opening, space, or cavity in a biological system).

The term “exsanguination” is the fatal process of total blood loss. As a cause of death in humans, exsanguination can arise in cases of trauma involving the rupturing of major blood vessels. If such injuries are not treated immediately, fatal blood loss may occur rapidly. For example, it is a common cause of battlefield deaths.

The term “septicemia” refers to the invasion of the bloodstream by virulent bacteria that multiply and discharge their toxic products. The disorder, which is serious and sometimes fatal, is commonly known as blood poisoning. The invasive organisms are usually Streptococci or Staphylococci but may be any type of bacteria.

As used herein “cold-packs” are two containers containing chemicals separated by a frangible seal. When the seal is broken, as the contents from the separate containers begin to react, energy is absorbed from the surroundings creating a cooling effect. An example of chemicals which can be mixed in a cold pack are ammonium nitrate and water. In certain embodiments the cold pack has two sealed bags, one inside the other. The outer bag is made of thick strong plastic. It contains a ammonium nitrate and the second plastic bag. The second (inner) bag is made of a thin weak plastic and contains water. When the bag is squeezed the inner bag breaks and the water mixes with the powder creating the cooling effect.

Contemplated equivalents of the polymers, subunits and other compositions described above include such materials which otherwise correspond thereto, and which have the same general properties thereof (e.g., biocompatible), wherein one or more simple variations of substituents are made which do not adversely affect the efficacy of such molecule to achieve its intended purpose. In general, the compounds of the present invention may be prepared by, for example, described below, or by modifications thereof, using readily available starting materials, reagents and conventional synthesis procedures. In these reactions, it is also possible to make use of variants which are in themselves known, but are not mentioned here.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Purification of Poloxamer 407

Poloxamer 407 (486.0 g, lot number WPHT-543B), purchased from BASF Corporation, Mount Olive, N.J., was dissolved in deionized water (15,733 g). The solution was maintained at 0.1° C. and 2335.1 g of (NH₄)₂SO₄ were added. The solution was equilibrated at 2° C. and after two distinct phases formed, the lower phase was discarded, and the upper phase (2060 g) was collected and weighed. Deionized water (14159 g) was added and the solution was equilibrated to 2° C. Next, 2171.6 g of (NH₄)₂SO₄ were added with stirring. After the salt was dissolved, the solution was maintained at approximately 2° C. until two phases formed. The upper phase (3340 g) was isolated and diluted with 12879 g of deionized water. The solution was chilled to about 2.2° C. and 2062 g of (NH₄)₂SO₄ were added. The phases were allowed to separate as above. The upper phase was isolated and extracted with 4 liters of dichloromethane. Two phases were allowed to form overnight. The organic (lower) phase was isolated and approximately 2 kg of sodium sulfate (Na₂SO₄) were added to it to remove the remaining water. The dichloromethane phase was filtered through a PTFE filter (0.45 μm pore size) to remove the undissolved salts. The dichloromethane was removed under vacuum at approximately 30° C. Final traces of dichloromethane were removed by drying in an oven overnight at about 30° C. A total of 297.6 g of fractionated poloxamer 407 (lot number 00115001) were recovered. The chemical and physical characteristics of the fractionated poloxamer 407 are compared to those of the starting material in Table 1, A “*” indicates a viscosity of a 25% solution measured at 30° C. using a cone and plate viscometer,

TABLE 1
Purification of Poloxamer 407.

| | | | | Unsaturation | Weight % | Viscosity, |
|---|---|---|---|---|---|---|
| Sample | M_{w} | Mₙ | M_{w}/Mₙ | MEq/g | oxyethylene | centipoise* |
| Poloxamer 407 | 11,996 | 9,979 | 1.20 | 0.048 | 73.2 | 275,000 |
| Poloxamer 407, | 13,551 | 12,775 | 1.06 | 0.005 | 69.3 | >820,000 |
| lot 00115001, | | | | | | |
| fractionated | | | | | | |

### Example 2

### In-Vitro Testing and Principal of Operation

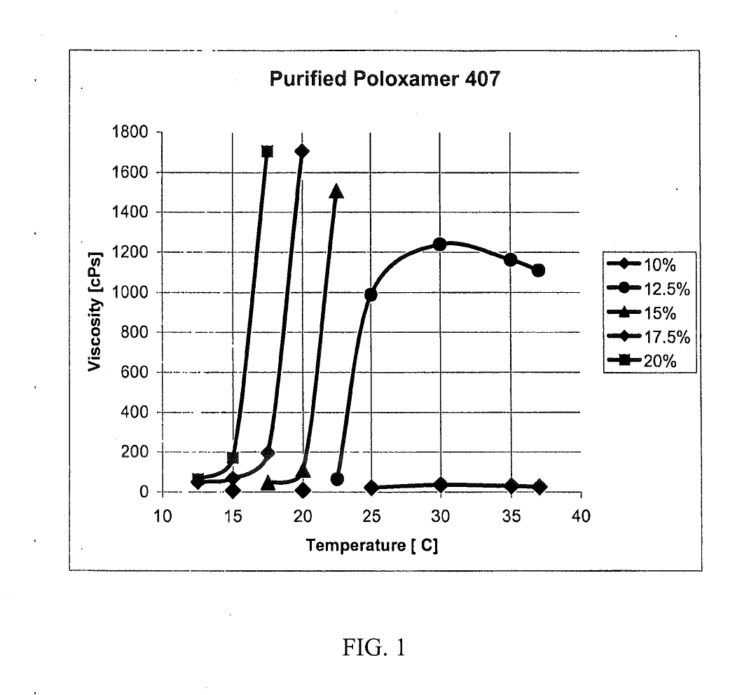

The viscosity changes were measured in a Brookfield Cone and Cup viscometer with temperature control. A graph of the viscosity changes (FIG. 1) clearly shows polymer concentrations from approximately 12.5 w % until at least 20 w % will show steep increases in solution viscosities with temperature. The onset of gelation is dependent on the temperature and higher polymer concentrations lead to earlier onsets of gelation. Furthermore, polymer concentrations below approximately 12.5 w % do not demonstrate an increase in solution viscosity with temperature and remain liquid even at body temperature.

These two findings demonstrate the potential operation principle of the purified poloxamer 407. The polymer solution is injected as a soft gel at the temperature of a typical OR (about 18° C.) into the arteriotomy and the rise in temperature leads to a stiff gel. The gel will start to dissolve in blood and when the concentration of the polymer decreases below approximately 12.5%, it turns back into a liquid, without any possibility to turn back into a gel at physiological temperatures. Alternatively, cooling of the gel with ice or cold saline would liquefy the gel as the temperature falls below the gelation point. As a liquid, it quickly dilutes in blood and again there is no possibility for it to turn back into a gel at physiological temperatures.

### Example 3

### Injectability of Purified Poloxamer 407 Through Various Needle Gauges

A three milliliter polycarbonate syringe (Merrit Medallion) was loaded in the cold with three milliliter of 20 w % purified poloxamer 407. Various sized needles were attached via a luer lock and the injectability of the polymer solution was tested at 6° C. (liquid state) and at room temperature (23° C.; soft gel state) as shown in the table below.

TABLE 2
Injectability of 20 w % purified poloxamer
407 through a 3 mL syringe.

| Needle | 6° C. | 23° C. |
|---|---|---|
| 16G | easy | easy |
| 18G | easy | easy |
| 21G | easy | easy |
| 25G | easy | pushable |
| 27G | easy | required hard push |

The same experiment was repeated using a one milliliter polycarbonate syringe (Merrit Medallion) and in all cases, the polymer could be easily injected through the various needle gauges.

TABLE 3
Injectability of 20 w % purified poloxamer
407 through a 1 mL syringe.

| Needle | 6° C. | 23° C. |
|---|---|---|
| 16G | easy | easy |
| 18G | easy | easy |
| 21G | easy | easy |
| 25G | easy | easy |
| 27G | easy | easy |

### Example 4

### Gelation Temperature of Selected Pluronic® and Tetronic® Polymer Solutions

The polymer was weighed into a plastic tube. To achieve the required concentration, the weight was multiplied by 4, for 25 weight percent (w %), and by 5, for 20 weight percent (w %), and the required final weight was achieved by adding saline. The solutions were placed in the fridge at 4° C. and usually were ready within 24 hours. Gelation points were measured in a Brookfield viscometer and the point at which viscosity exceeded the range of the plate/cone (greater than about 102,000 cP) was called the gelation temperature.

TABLE 4
Gelation Temperature of Selected Inverse
Thermosensitive Polymers in Saline.

| polymer | concentration | temperature |
|---|---|---|
| Tetronic 1107 | 25 w % | 27° C. |
| Tetronic 1107 | 20 w % | 34° C. |
| Purified Tetronic 1107 | 25 w % | 22° C. |
| Purified Tetronic 1107 | 20 w % | 32.5° C. |
| Tetronic 1307 | 25 w % | 24.5° C. |
| Tetronic 1307 | 20 w % | 31° C. |
| Purified Tetronic 1307 | 25 w % | 20° C. |
| Purified Tetronic 1307 | 20 w % | 26° C. |
| Pluronic F108 | 25 w % | 26° C. |
| Pluronic F108 | 20 w % | 60° C. |
| Purified Pluronic F108 | 25 w % | 19° C. |
| Purified Pluronic F108 | 20 w % | 26° C. |

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of temporarily occluding a bleeding internal wound cavity in a mammal, comprising:
directly introducing into said bleeding internal wound cavity a composition comprising at least one purified inverse thermosensitive polymer, and
forming a gel in said bleeding internal wound cavity sufficient to temporarily occlude said bleeding internal wound cavity.

2. The method of claim 1, wherein said at least one purified inverse thermosensitive polymer is selected from the group consisting of purified poloxamers and purified poloxamines.

3. The method of claim 1, wherein said at least one purified inverse thermosensitive polymer is selected from the group consisting of purified poloxamer 407, purified poloxamer 338, purified poloxamer 118, purified poloxamine 1107, and purified poloxamine 1307.

4. The method of claim 1, wherein said composition further comprises a therapeutic agent.

5. The method of claim 1, wherein said composition further comprises a therapeutic agent; and the therapeutic agent is selected from the group consisting of antiinflammatories, antibiotics, antimicrobials, chemotherapeutics, antivirals, analgesics, and antiproliferatives.

6. The method of claim 1, wherein said composition is introduced to said bleeding internal wound cavity using a syringe, cannula, tube, packet, or catheter.

7. The method of claim 1, further comprising the step of cooling said composition prior to introduction into said bleeding internal wound cavity.

8. A method of temporarily occluding a bleeding internal wound cavity in a mammal, comprising: placing an elastomeric balloon into said bleeding internal wound cavity;
directly introducing into the balloon, a composition comprising at least one purified inverse thermosensitive polymer, and
forming a gel in the balloon, thereby inflating said balloon and temporarily occluding said bleeding internal wound cavity.
